(19) United States
(12) Patent Application Publication
(10) Pub. No.: US 2025/0171766 A1
(43) Pub. Date: May 29, 2025

(54) DIGITAL COUNTING OF CELL FUSION EVENTS USING DNA BARCODES

(71) Applicant: A-Alpha Bio, Inc., Seattle, WA (US)

(72) Inventors: Ryan Emerson, Seattle, WA (US); Randolph Lopez, Seattle, WA (US); Emily Engelhart, Seattle, WA (US); David Younger, Seattle, WA (US)

(21) Appl. No.: 18/833,072

(22) PCT Filed: Jan. 27, 2023
(86) PCT No.: PCT/US2023/011768

§ 371 Date: Jul. 25, 2024

Related U.S. Application Data
(60) Provisional application No. 63/304,380, filed on Jan. 28, 2022.

(51) Int. Cl. C12N 15/10

(52) U.S. Cl. CPC C12N 15/1037; C12N 15/1065; C12N 15/1082

(57) ABSTRACT

Compositions and methods for estimating the number of cell fusion events that occur in a liquid culture using multiplexed oligonucleotide molecular barcodes and next-generation sequencing are disclosed. A method for quantifying unique cell fusion events, the method comprising: providing a first quantity of cells, wherein each cell of the first quantity of cells comprises an exogenous nucleic acid vector of a first library of exogenous nucleic acid vectors.

### CROSS-REFERENCE TO RELATED APPLICATIONS

This application claims the benefit of U.S. Provisional Patent Application No. 63/304,380, filed on Jan. 28, 2022. The contents of this application are incorporated herein by reference in their entirety.

### FEDERALLY SPONSORED RESEARCH OR DEVELOPMENT

This invention was made with government support under contract #1950992 awarded by the National Science Foundation. The Government has certain rights in the invention.

### FIELD OF THE INVENTION

This disclosure relates to quantifying cell fusion events in liquid culture using multiplex DNA barcodes and can be used, for example, to improve the accuracy of high-throughput assays for identifying and measuring protein-protein interactions.

### BACKGROUND

Identifying and quantifying the strength of protein-protein interactions (PPIs) between protein binding partners and characterizing complex PPI networks is a central goal for biomedical research and development, and high-throughput methods directed to characterizing PPI networks may be useful for drug discovery, protein engineering, characterizing receptor-ligand binding dynamics, among other applications. Protein binding partners may include, for example, a ligand and its receptor, an antibody and its antigen, an E3 ubiquitin ligase and its substrate, among many other examples of protein binding partners. Various high-throughput methods including yeast two-hybrid screening, affinity purification coupled to mass spectrometry, phage, and yeast surface display methods, among others have been developed to interrogate PPI networks. For all these methods, accurately identifying the protein binding partners and quantifying the binding affinity between the protein binding partners, along with increasing throughput and decreasing costs of the assay, are desirable features that may be optimized.

Another approach, based on synthetic yeast agglutination, relies on reprogramming yeast sexual agglutination—a naturally-occurring protein-protein interaction—to link protein-protein interaction strength with mating efficiency between a-type recombinant haploid yeast cells and α-type recombinant haploid yeast cells in liquid culture (see, e.g., U.S. Pat. No. 11,136,573). For a PPI screening platform based on synthetic yeast agglutination, mating efficiency, represented by the number of diploid yeast cells formed in a turbulent liquid culture, is a proxy for PPI affinity. Therefore, the accuracy of the PPI screening platform depends on accurately reconstructing the number of diploid yeast cells formed over the course of the liquid-culture based assay from the end-point readout.

### SUMMARY

The compositions and methods disclosed herein are based, at least in part, on the discovery that a multiplexed oligonucleotide molecular barcoding approach can be used to estimate the number of cell-cell fusion events in a liquid culture more accurately. For example, the multiplexed barcoding approach can be used to estimate the number of diploid formation events in a liquid culture of haploid yeast cells. The multiplexed barcoding approach also can be used to estimate the number of diploid formation events in a PPI screening platform based on yeast synthetic agglutination in liquid culture. For a library-by-library screen of PPIs, a library of proteins of interest (POIs), or variants thereof, may be screened for interaction against another library of POIs, or variants thereof, according to the synthetic yeast agglutination compositions and methods disclosed herein. The compositions and methods described herein provide increased accuracy in detecting diploid formation events for PPI screening platforms based on synthetic yeast agglutination.

A pairing of protein binding partners is referred to herein as a POI_{A}-POI_{α} pair, with the proteins being expressed by an a-type recombinant haploid yeast cell and an α-type recombinant haploid yeast cell, respectively. Applicants have discovered that during POI library construction, instead of assigning a single unique oligonucleotide molecular barcode to a specific POI, each POI can be combined with a plurality of unique oligonucleotide molecular barcodes of a sufficient number such that a substantial majority of POI_{A}-POI_{α} diploid formation events during subsequent agglutination assays will each comprise a unique barcode-barcode combination.

Regardless of experimental variation introduced in subsequent steps of the PPI screening platform, the observed number of unique barcode-barcode combinations with any sequencing support from a given POI_{A}-POI_{α} interaction compared to the number of possible barcode-barcode combinations from that POI_{A}-POI_{α} interaction can then be used to provide a highly accurate estimate of the number of diploid formation events that occurred during the liquid culture yeast synthetic agglutination assay.

Described herein are methods for quantifying unique cell fusion events. The methods include providing a first quantity of cells, wherein each cell of the first quantity of cells comprises an exogenous nucleic acid vector of a first library of exogenous nucleic acid vectors, wherein each of the exogenous nucleic acid vectors in the first library comprises a first open reading frame (ORF) linked to an oligonucleotide molecular barcode sequence selected from a first plurality of oligonucleotide molecular barcode sequences. The methods further include providing a second quantity of cells, wherein each cell of the second quantity of cells comprises an exogenous nucleic acid vector of a second library of exogenous nucleic acid vectors, wherein each of the exogenous nucleic acid vectors in the second library comprises a second ORF linked to an oligonucleotide molecular barcode sequence selected from a second plurality oligonucleotide molecular barcode sequences.

The methods further include combining the first quantity of cells and the second quantity of cells in a liquid medium to produce a culture. The methods further include growing the culture for a time and under conditions sufficient to enable fusion events to occur between cells of the first quantity of cells and cells of the second quantity of cells to produce a plurality of fused cells, wherein a recombination event occurs between the first exogenous nucleic acid vector and the second exogenous nucleic acid vector within the fused cells to produce combined oligonucleotide molecular barcode sequences.

The methods further include sequencing combined oligonucleotide molecular barcode sequences from the culture, determining, for each pair of first and second ORF, a first number of unique pairs of first and second oligonucleotide molecular barcode sequences within the combined oligonucleotide molecular barcodes observed in the culture, determining, for each pair of first and second ORF, a second number of possible combined oligonucleotide molecular barcode sequences, and calculating an estimated number of unique fusion events in the culture based on the first number and second number.

In some embodiments, the first quantity of cells and the second quantity of cells are yeast cells. In some embodiments, the first quantity of cells comprise a-type haploid yeast cells and the second quantity of cells comprises α-type haploid yeast cells. In some embodiments, the first ORF encodes a protein of interest “a” (POIa) and the second ORF encodes a protein of interest “α” (POIα).

In other embodiments, each ORF encoding a POI_{α} is operably linked to an oligonucleotide molecular barcode sequence selected from the first plurality of oligonucleotide molecular barcode sequences and each ORF encoding a POIα is operably linked to an oligonucleotide molecular barcode sequence selected from the second plurality of oligonucleotide molecular barcode sequences.

In some embodiments, each POIa is expressed on the surface of a cell of the first quantity of cells and each POIα is expressed on the surface of a cell of the second quantity of cells. In some embodiments, at least one of the first quantity of cells or the second quantity of cells has been rendered incapable of mating according to any native sexual agglutination process such that the first quantity of recombinant haploid yeast cells and the second quantity of recombinant haploid yeast cells are not capable of mating according to any native sexual agglutination process.

In some embodiments, each POIa and each POIα are synthetic adhesion proteins (SAPs). In certain embodiments, each POIa and each POIα are either i) a fusion protein bound to a cell wall glycosylphosphatidylinositol (GPI) anchored protein residing on a surface of a portion of the first quantity of recombinant haploid yeast cells or the second quantity of haploid yeast cells; or ii) a glycosylphosphatidylinositol (GPI) anchored fusion protein residing on the surface of a portion of the first quantity of haploid yeast cells or the second quantity of haploid yeast cells.

In other embodiments, the first plurality of oligonucleotide molecular barcode sequences comprises three or more unique oligonucleotide molecular barcode sequences and/or the second plurality of oligonucleotide molecular barcode sequences comprises three or more oligonucleotide molecular barcode sequences. In some embodiments, the first plurality of oligonucleotide molecular barcode sequences comprises 10 or more unique oligonucleotide molecular barcode sequences and/or the second plurality of oligonucleotide molecular barcode sequences comprises 10 or more oligonucleotide molecular barcode sequences. In other embodiments, the first plurality of oligonucleotide molecular barcode sequences comprises 100 or more unique oligonucleotide molecular barcode sequences and/or the second plurality of oligonucleotide molecular barcode sequences comprises 100 or more oligonucleotide molecular barcode sequences. In other embodiments, the first plurality of oligonucleotide molecular barcode sequences comprises 1000 or more unique oligonucleotide molecular barcode sequences and/or the second plurality of oligonucleotide molecular barcode sequences comprises 1000 or more oligonucleotide molecular barcode sequences.

In some embodiments, the second number of possible oligonucleotide molecular barcode pairs is 7, 8, 9, 10, or greater. In other embodiments, the second number of possible oligonucleotide molecular barcode pairs is 100 or greater. In other embodiments, the second number of possible oligonucleotide molecular barcode pairs is 10,000 or greater.

In other embodiments, the library of POIₐs comprises 10 or more POIₐs and/or the library of POI_{α}s comprises 10 or more POI_{α}s. In other embodiments, the library of POIₐs comprises 100 or more POIₐs and/or the library of POI_{α}s comprises 100 or more POI_{α}s. In other embodiments, the library of POIₐs comprises 1000 or more POIₐs and/or the library of POIαs comprises 1000 or more POIαs. In other embodiments, the library of POIₐs comprises 10,000 or more POIₐs and/or the library of POI_{α}s comprises 10,000 or more POI_{α}s.

In some embodiments, the first exogenous nucleic acid vector and the second exogenous nucleic acid vector each further comprise a unique primer binding site, a recombination site, and a selectable marker. In some embodiments, each cell of the first quantity of cells and each cell of the second quantity of cells further comprises an exogenous recombinase. In some embodiments, the exogenous recombinase mediates the recombination event.

In some embodiments, sequencing a portion of the first oligonucleotide molecular barcode sequence and a portion of the second oligonucleotide molecular barcode sequence yields a plurality of sequencing reads, each sequencing read comprising a portion of the first oligonucleotide molecular barcode sequence and a portion of the second oligonucleotide molecular barcode sequence.

In some embodiments: i) each cell of the first quantity of cells lacks either a functional Aga1 or a functional Aga2 protein, and/or ii) each cell of the second quantity cells lacks a functional Sagl protein.

The term “complementary nucleotides” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10, or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TTAGCTGG-3′.

The terms “homology,” “identity,” or “similarity” as used herein with respect to sequences refer to sequence similarity between two strands of amino acids, e.g., peptides or proteins, or strands of nucleotides or bases, e.g., nucleic acid molecules. The terms “homologous region” or “homology arm” refer to a region on a donor DNA with a certain degree of homology with a target genomic DNA sequence. Homology can be determined by comparing a position in each sequence that is aligned for purposes of comparison. When a position in the compared sequence is occupied by the same base or amino acid, then the molecules are homologous at that position. A degree of homology between sequences is a function of the number of matching or homologous positions shared by the sequences.

“Operably linked” as used herein refers to an arrangement of elements, e.g., barcode sequences, gene expression cassettes, coding sequences, promoters, enhancers, transcription factor binding sites, where the components so described are configured so as to perform their usual function. Thus, control sequences operably linked to a coding sequence are capable of effecting the transcription, and in some cases, the translation, of a coding sequence. The control sequences need not be contiguous with the coding sequence as long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

As used herein the term “selectable marker” refers to a gene introduced into a cell, which confers a trait suitable for artificial selection. General use selectable markers are well known to those of ordinary skill in the art. Drug selectable markers such as ampicillin/carbenicillin, kanamycin, chloramphenicol, erythromycin, tetracycline, gentamicin, bleomycin, streptomycin, puromycin, hygromycin, blasticidin, and G418 can be employed. A selectable marker can also be an auxotrophy selectable marker, wherein the cell strain to be selected carries a mutation that renders it unable to synthesize an essential nutrient. Such a strain will grow only if the lacking essential nutrient is supplied in the growth medium. Essential amino acid auxotrophic selection of, for example, yeast mutant strains, is common and well known in the art. “Selective medium” as used herein refers to a cell growth medium to which has been added a chemical compound or biological moiety that selects for or against selectable markers or a medium that is lacking essential nutrients and selects against auxotrophic strains.

As used herein, the term “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, bacterial artificial chromosomes (BACs), yeast artificial chromosomes (YACs), P1-derived artificial chromosomes (PACs), and synthetic chromosomes, among others.

As used herein, “affinity” is the strength of a binding interaction between a biomolecule and its ligand or binding partner. Affinity is usually measured and described using the equilibrium dissociation constant, K_{D}. The lower the K_{D} value, the greater the binding affinity. Affinity may be affected by hydrogen bonding, electrostatic interactions, hydrophobic and Van der Waals forces between the binding partners, or by the presence of other molecules, e.g., binding agonists or antagonists.

In some implementations, affinity may be described using arbitrary units, wherein a certain binding affinity within an assay, for example, the binding affinity between two wild-type protein binding partners or the wild-type species of a first protein binding partner and the wild-type species of a second protein binding partner, is set to an arbitrary unit of 1.0 and binding affinities for other pairs of protein binding partners, for example, the mutant species of a first protein binding partner and the mutant species of a second protein binding partner, are measured relative to that certain binding affinity.

As used herein, “site saturation mutagenesis” (SSM), refers to a mutagenesis technique used in protein engineering and molecular biology, wherein a codon or set of codons is substituted with most or all possible amino acids at the position in the polypeptide. SSM can be performed for one codon, several codons, or for every position in the polypeptide. Substitutions can be performed to all possible alternative amino acids or select amino acids can be omitted. For example, substitutions to cysteine are often omitted due to deleterious effects on yeast surface expression and protein folding. The result is a library of mutant proteins representing multiple single-residue amino acid substitutions at one, several, or every amino acid position in a polypeptide.

As used herein, “user-directed mutagenesis” refers to any process wherein a user modifies the amino acid sequence of a polypeptide encoded by a polynucleotide (nucleic acid molecule) by modifying the polynucleotide sequence. A polypeptide sequence can be modified by user-directed mutagenesis of the polynucleotide sequence that encodes the polypeptide. A polypeptide can be modified at one or more amino acid residues in a defined way, e.g. an alanine residue may be changed to an arginine residue, or a polypeptide may be modified in a randomized way, i.e., by using degenerate primers and randomized PCR amplification to modify the polynucleotide sequence that encodes the polypeptide. A polypeptide can be modified by user-directed mutagenesis at one amino acid residue or many amino acid residues. A polypeptide can be modified by user-directed mutagenesis such that an amino acid residue at a given position is modified to one of a subset of possible amino acid substitutions at the position, for example, a conservative amino acid substitution as is known in the art, or a substitution to all possible amino acids except for cysteine. A polypeptide can be modified by user-directed mutagenesis of the polynucleotide sequence that encodes the polypeptide to include insertion and/or deletions of one or more amino acid residues, or a polypeptide sequence can be truncated by user-direction mutagenesis. A polypeptide can be modified by user-directed mutagenesis to include insertions or substitutions with natural or unnatural amino acids.

As used herein, the term “protein of interest” (“POI”) refers to a polypeptide molecule, the biochemical properties of which are the subject of interrogation by the compositions and methods disclosed herein. A POI may be a full-length protein, a truncated protein, a fusion protein, or a functionally tagged protein, among other species and variants of proteins. In some implementations, a first POI or library of variants thereof is screened for binding affinity against a second POI or library of variants thereof. In some implementations, a first POI is expressed by an a-type haploid yeast cell and may be referred to as a “POI_{A}” and a second POI is expressed by an α-type haploid yeast cell and may be referred to as a “POI_{α}.” In some implementations, where an interaction is detected between a POI_{A} and a POI_{α} by the compositions and methods disclosed herein, the first POI and the second POI may be referred to as a “POI_{A}-POI_{α} pair.”

As used herein, “protein-protein interaction” (“PPI”) refers to physical contacts of high specificity established between two or more proteins as a result of biochemical events driven by electrostatic forces including, for example, a hydrophobic effect. Many protein-protein interactions are physical contacts between the surfaces of each of the proteins, with molecular associations between specific domains of the proteins that occur in a cell or in a living organism in a specific biomolecular context. In some implementations, the protein-protein interactions are strong enough to replace the function of the native sexual agglutination proteins. For example, it is possible to couple mating efficiency to the interaction strength of a particular protein-protein interaction. In certain embodiments, the assay can characterize or determine protein-protein interactions between synthetic adhesion proteins. In certain embodiments, a protein-protein interaction is modulated, either strengthened or inhibited, by a third chemical entity, which could be a small molecule, polypeptide, or polynucleotide, among others.

As used herein, a “synthetic adhesion protein” (“SAP”) refers to any protein or polypeptide to be assayed for binding to or interacting with any other any protein or polypeptide. The proteins can be expressed heterologously or exogenously. Synthetic adhesion proteins are referred to as such, because they are not typically associated with the adhesion required for agglutination as in wild type sexual agglutination proteins.

As used herein, “mediate” means to promote or catalyze a process, for example, a recombinase can mediate recombination between double-stranded or single-stranded polynucleotides. As another example, sexual agglutination proteins expressed on the surface of yeast cells can mediate agglutination and subsequent cellular fusion between haploid yeast cells of opposite mating types.

The compositions and methods disclosed herein provide several advantages. For the PPI screening platform based on yeast synthetic agglutination in liquid culture, the key event being detected is the formation of diploid yeast cells mediated by the interaction of a POI_{A} expressed on the surface of an a-type recombinant haploid yeast cell and a POI_{α} expressed on the surface of an α-type recombinant haploid yeast cell. The number of diploid formation events, i.e., mating efficiency between a-type haploids and α-type haploids, is a proxy for the affinity between a POI_{A} and a POI_{α}. Indeed, mating efficiency and POI_{A}-POI_{α} are related log-linearly across over five orders of magnitude of K_{D} (see, Younger et al., “High-throughput characterization of protein-protein interactions by reprogramming yeast mating,” PNAS USA, 14;114 (46): 12166-12171 (2017)). However, after diploid formation events in liquid culture occur over time, several subsequent processes contribute stochastic or systematic variation to the eventual quantitative output and degrade the quantitative accuracy of the estimation of affinity for a given POI_{A}-POI_{α} pair. For example, the expression of some proteins in yeast cells may result in a greater metabolic load that other proteins, causing diploid yeast cells that express those proteins to grow more slowly than diploid yeast cells expressing other proteins. Stochastic or systematic differences among diploid yeast cells contribute to variation in quantifying the number of fusion events in the assay.

Sources of stochastic or systematic variation may include (1) the time at which a cell fusion occurs over the course of an assay that is longer than 90 minutes (2) growth rate differences of diploid yeast cells in liquid culture over the course of a greater than 90 minute assay; (3) amplification biases or stochastic variation in amplification rate during PCR amplification of unique recombined barcode-barcode pairs; and/or (4) next-generation sequencing (NGS) library preparation of PCR-amplified barcode-barcode pairs. The output of these processes yields NGS sequencing reads for a given barcode-barcode pair, the abundance of which is an indirect estimate of the number of diploid formation events mediated by the corresponding POI_{A}-POI_{α} interaction. However, this quantitative readout is susceptible to the sources of variation described above.

The compositions and methods disclosed herein, i.e., utilizing a plurality of unique oligonucleotide molecular barcodes for each POI rather than a single barcode per POI, obviate the sources of stochastic and systematic variation described above and substantially improve the quantitative accuracy of the estimation of PPI affinity for a measured POI_{A}-POI_{α} interaction. The result is, in effect, a “digital” readout such that the detection of a unique barcode-barcode sequence in the NGS readout of the platform represents a unique diploid formation event, regardless of the abundance of sequencing reads corresponding to that unique barcode-barcode combination. The number of unique barcode-barcode sequences detected for a POI_{A}-POI_{α} pair, rather than the abundance of sequencing reads associated with that POI_{A}-POI_{α} pair, represents the number of diploid formation events during the assay and is used to infer PPI affinity for the POI_{A}-POI_{α} interaction.

Unless otherwise defined, all technical and scientific terms used herein have the same meaning as commonly understood by one of ordinary skill in the art to which these compositions and methods belong. Although compositions and methods similar or equivalent to those described herein can be used in the practice or testing of the compositions and methods disclosed herein, suitable compositions and methods are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively, unless expressly limited. Additionally, the words “herein,” “above,” and “below” and words of similar import, when used in this application, shall refer to this application as a whole and not to any particular portions of the application.

While disclosures have been particularly shown and described herein with reference to various alternate aspects, it will be understood by persons skilled in the relevant art that various changes in form and details can be made herein without departing from the spirit and scope of the invention. The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic diagram of natural and synthetic yeast agglutination in S. cerevisiae.

FIG. 2A is a schematic diagram of the recombination between SAP expression cassettes mediated by exogenous Cre recombinase.

FIG. 2B is more detailed schematic of the recombination between SAP expression cassettes mediated by exogenous Cre recombinase.

FIG. 2C is a schematic diagram of the recombination between SAP expression cassettes mediated by exogenous Cre recombinase indicating PCR amplification of the unique barcode-barcode pair that is a result of the diploid formation event and subsequent recombination of the SAP expression cassettes.

FIG. 3 is a schematic diagram of a yeast synthetic agglutination assay for a POI_{A}-POI_{α} pair where each POI is linked to a single oligonucleotide barcode species.

FIG. 4 is a schematic diagram of a yeast synthetic agglutination assay for a POI_{A}-POI_{α} pair where each POI is linked to a plurality of oligonucleotide molecular barcode species.

FIG. 5A is a schematic diagram of portions of nucleic acid constructs where an ORF encoding a POI was synthesized with a plurality of oligonucleotide molecular barcode sequences, with each ORF being linked to a different unique oligonucleotide molecular barcode sequence.

FIG. 5B is a schematic diagram of portions of nucleic acid constructs where a library of oligonucleotide molecular barcode sequences was synthesized separately and assembled with the ORF encoding a POI by isothermal in vitro assembly, yielding a plurality of nucleic acid constructs, each comprising ORF encoding a POI with each ORF being linked to a different unique oligonucleotide molecular barcode sequence.

FIG. 6 is a histogram plot of the frequency of ‘possible’ and ‘observed’ barcode-barcode combinations for POI_{A}-POI_{α} pairs.

FIG. 7 is a histogram plot of the distribution of sequencing reads for POI_{A}-POI_{α} pairs where 10 diploid yeast were formed during the synthetic agglutination assay.

FIG. 8 is graph of the distribution of estimated diploids for POIA-POIα pairs that have an estimated 10 diploid formation events during the synthetic agglutination assay, compared to a Poisson distribution of expected values.

FIG. 9 is a plot of a comparison of confidence interval calibration with or without multiplexed barcoding across POI_{A}-POI_{α} networks of various sizes. Like reference symbols in the various drawings indicate like elements.

### DETAILED DESCRIPTION

Synthetic Yeast Agglutination with Multiplexed Barcodes

The present disclosure provides methods for highly accurate estimation of PPI affinity by improving on the accuracy of the proxy of sequencing read depth for protein-protein interaction PPI intensity by replacing read depth with an estimate of the number of diploids formed. Synthetic yeast agglutination relies on reprogramming yeast sexual agglutination—a naturally-occurring protein-protein interaction—to link protein-protein interaction strength with mating efficiency between a-type recombinant haploid yeast cells and α-type recombinant haploid yeast cells in liquid culture. For a PPI screening platform based on synthetic yeast agglutination, mating efficiency, represented by the number of diploid yeast cells formed in a turbulent liquid culture, is a proxy for PPI affinity. Therefore, the accuracy of the PPI screening platform depends on accurately reconstructing the number of diploid yeast cells formed over the course of the liquid-culture based assay from the end-point readout. The compositions and methods disclosed herein provide significantly increased accuracy in detecting diploid formation events for a PPI screening platform based on synthetic yeast agglutination.

As discussed in further detail below, for each POI in the library of POIs, a plurality of unique oligonucleotide molecular barcodes are assigned to a single open reading frame (ORF) encoding a POI within the library of POIs. A sufficient number of unique barcodes are assigned to each POI such that the number of possible barcode-barcode combinations is substantially more than the expected number of diploids formed in a given assay, even for a strong PPI where many diploid formation events are expected. A substantial majority of diploid formation events will form unique barcode combinations, identifiable by sequencing. Rather than quantifying diploid formation based on sequencing read depth of a single barcode-barcode combination that represents a single POI_{A}-POI_{α} pair, the method provided herein quantify the number of observed unique barcode combinations to represent the number of diploids formed for that POI_{A}-POI_{α} pair. This quantity is only minimally affected by yeast cell growth conditions, PCR amplification, or NGS library prep and therefore provides a better estimate of diploid formation events than can be derived from sequencing read depth alone.

For example, if a diploid formation event occurs at hour 7 of a 16 hour synthetic agglutination assay in liquid culture, the resulting barcode combination will be unique and quantified equivalently to a barcode resulting from a diploid formation event that occurs at hour 1, despite the fact that sequencing reads of the hour 1 barcode may vastly outnumber sequencing reads of the hour 7 barcode. Given that the doubling rate for yeast haploid and diploid cells is approximately 90 minutes, in the 6 hours between hour 1 and hour 7, the diploid cell that was formed by a fusion event at hour 1 would be expected to undergo 4 doublings, resulting in 2\hat{ }4 cells or 16 cells. Without the multiplexed barcoding methods disclosed herein, a diploid formation event at hour 1 would be counted approximately 16 times compared to a diploid formation event at hour 7. The multiplexed barcoding methods disclosed herein provide a more accurate estimate of the number of fusion events by controlling for this source of variation and counting fusion events by the presence or absence of unique barcode-barcode pairs formed in cell-cell fusion events.

In prior methods, e.g., as disclosed in U.S. Pat. No. 10,988,759 and 11,136,573, each POI was assigned a unique oligonucleotide molecular barcode, and after diploid formation events, these protein-specific barcodes were recombined and sequenced to identify the individual synthetic adhesion proteins (SAPs) that had mediated the corresponding diploid formation event. Quantifying sequencing reads of unique barcode-barcode combinations acted as a proxy measure of the number of diploid formation events, and thus, PPI affinity.

Replacing Native Agglutination Proteins with Multiplex Barcoded POIs

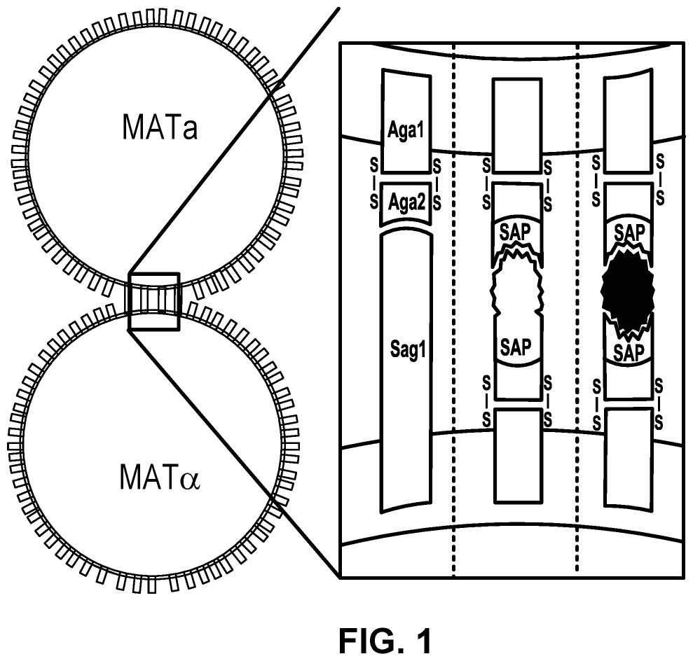

FIG. 1 shows a schematic depiction of natural and synthetic sexual agglutination in S. cerevisiae. At the left, the MATa and MATα haploids are shown at the top and bottom, respectively. The cell wall of each haploid cell is shown in grey. In a turbulent liquid culture, MATa and MATα haploid cells stick to one another due to the binding of sexual agglutinin proteins, which allows them to mate. The native sexual agglutinin proteins consist of Agal and Aga2, expressed by MATa cells, and Sagl, expressed by MATα cells. Agal and Sagl form glycosylphosphatidylinositol (GPI) anchors with the cell wall and extend outside of the cell wall with glycosylated stalks (see left frame of inset). Aga2 is secreted by MATa cells and forms a disulfide bond with Agal. The interaction between Aga2 and Sag1 is essential for wild-type sexual agglutination. The native sexual agglutinin interaction can be replaced with an engineered one by expressing Agal in both mating types and fusing complementary binders to Aga2 (see middle frame of inset). Instead of direct agglutination, it may be possible to express binders for a multivalent target, such that agglutination and mating only occurs in the presence of the target (see right frame of inset).

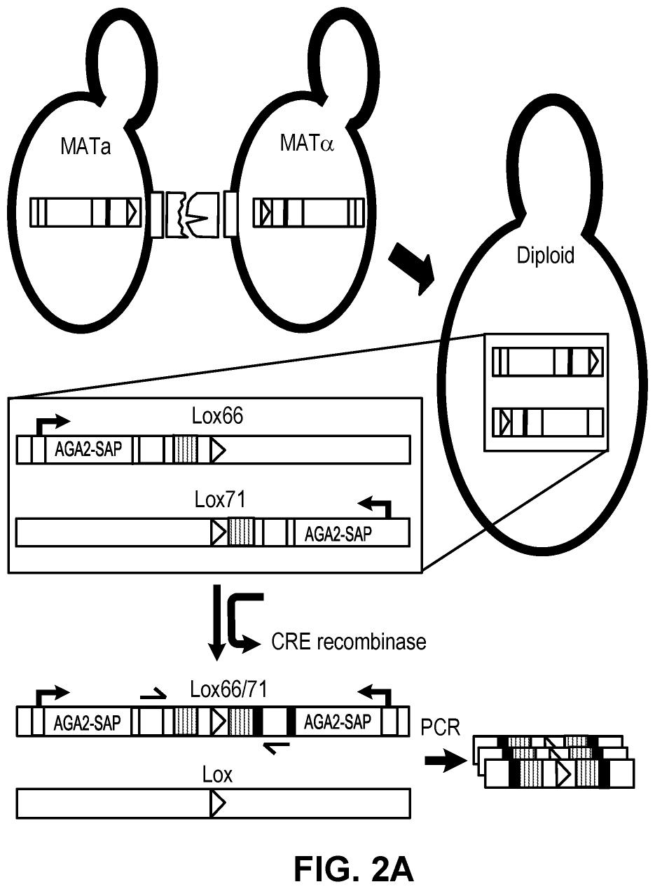

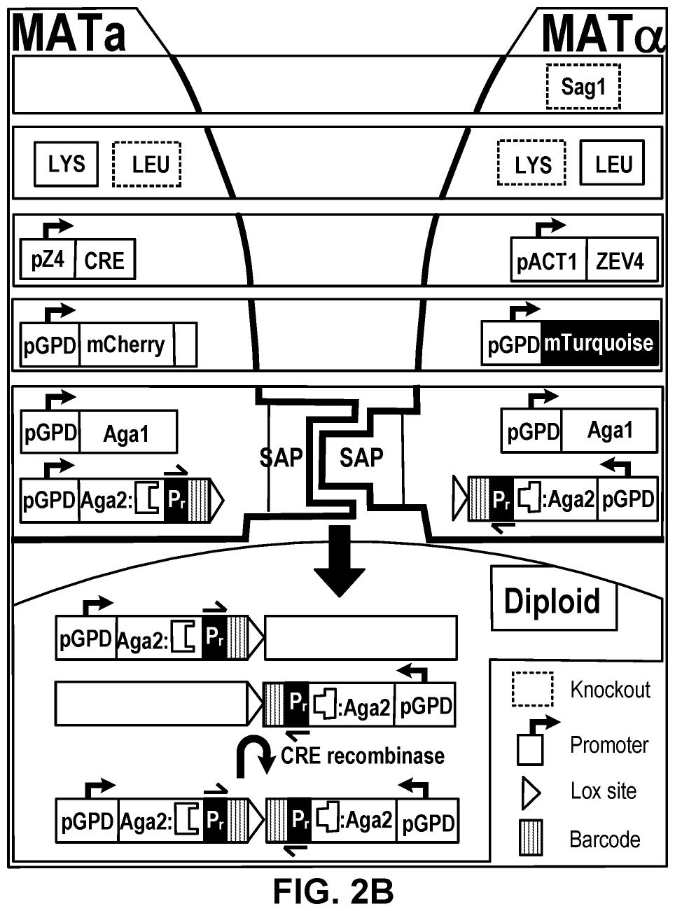

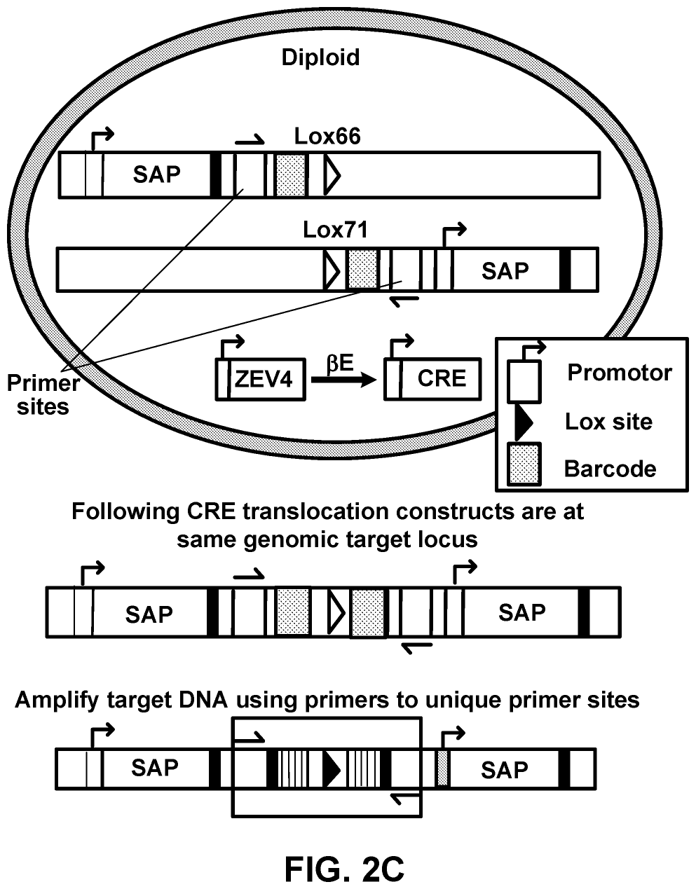

FIG. 2A shows a schematic of the Cre recombinase translocation scheme for high throughput analysis of display pair interactions. Here, a mating between a single recombinant MATa yeast strain and a single recombinant MATa yeast strain is shown. For a batched mating assay, however, a library of displayer cells of each mating type would be used (each comprising a library of SAPs fused to Aga2). Each MATa and MATα haploid cell contains a SAP fused to Aga2 integrated into a target chromosome (for example, chromosome III). Upon mating, both copies of the target chromosome are present in the same diploid cell.

In addition to the SAP/Aga2 cassette, each copy of the target chromosome has a unique primer binding site, one of a plurality of unique oligonucleotide barcodes operably linked to the particular SAP, and a lox recombination site. The plurality of oligonucleotide barcodes can be synthesized and assembled with the library of SAP expression cassettes such that a single SAP species is operably linked to a plurality of unique oligonucleotide barcodes. Upon expression of Cre recombinase, a chromosomal translocation occurs at the lox sites, resulting in a juxtaposition of the primer binding sites and barcodes onto the same copy of the target chromosome. A PCR is then performed to amplify a region of the chromosome containing the barcodes from both SAPs, such that sequences comprising unique barcode-barcode pairs, each representing a diploid formation event, are amplified.

In a batched mating, the result is a pool of fragments, each containing the unique barcode-barcode pair associated with two SAPs that were responsible for the single diploid formation event. Paired-end next generation sequencing is then used to match the barcodes and determine the number of diploid formation events mediated by that SAP pair.

FIG. 2B shows another schematic of the Cre recombinase translocation scheme for high throughput analysis of display pair interactions. The a-agglutinin, Sag 1, is knocked out in MATa cells to eliminate native agglutination. MATa and MATalpha cells are able to synthesize lysine or leucine, respectively. Diploids can then be selected for in media lacking both amino acids. MATa cells express ZEV4, a βE inducible transcription factor that activates Cre recombinase expression in diploid cells. MATa and MATalpha cells express mCherry and mTurquoise, respectively, for identification of strain types with flow cytometry. MATa and MATalpha cells constitutively express Agal along with a uniquely barcoded SAP fused to Aga2. When Cre recombinase expression is induced in diploids with βE, a chromosomal translocation at lox sites consolidates both SAP-Aga2 fusion expression cassettes onto the same chromosome. A single fragment containing the unique barcode-barcode sequence associated with that diploid formation event is then amplified by PCR with primers annealing to P_{f} and Pᵣ (primers specific to the primers from the first and second nucleic acid constructs integrated at the genomic target site) and sequenced to quantify the number of diploid formation events and identify the interacting SAP pair.

FIG. 2C shows a schematic of the CRE recombinase translocation scheme for high throughput analysis for interactions between SAPs from a library to library screen. When CRE recombinase expression is induced in diploids with βE, a chromosomal translocation at lox sites consolidates both SAP-Aga2 expression cassettes onto the same chromosome. A single fragment containing the unique barcode-barcode sequence associated with that diploid formation event is then amplified by PCR with primers annealing to primer binding sites from each of the first and second nucleic acid constructs and sequenced (for example, using a paired end analysis of next generation sequencing) to quantify the number of diploid formation events and identify the interacting SAP pair.

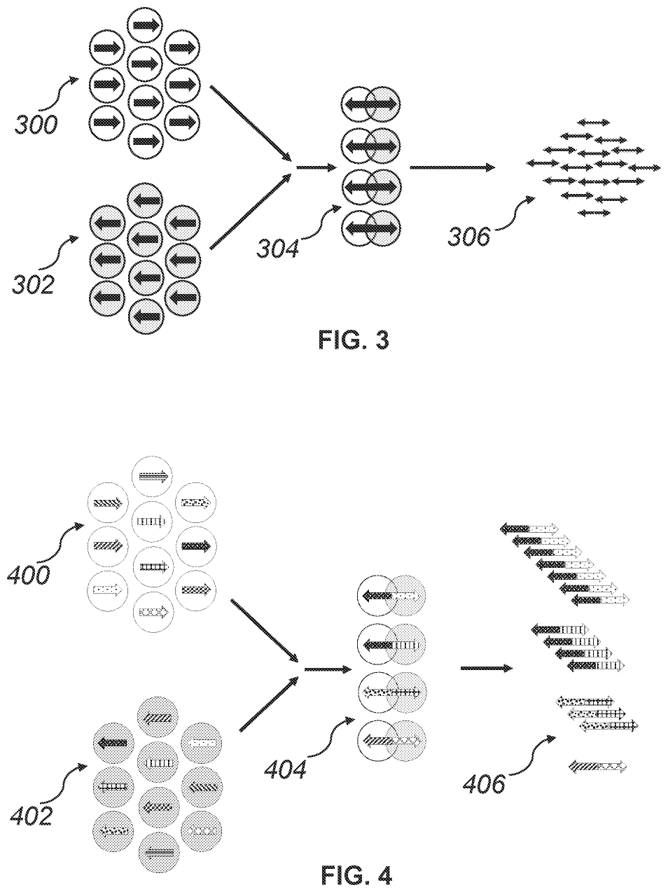

FIG. 3 is a schematic of a yeast synthetic agglutination assay for a POI_{A}-POI_{α} pair without multiplexed barcoding, i.e., each POI is linked to a single oligonucleotide barcode species. Yeast cell population 300 is a population of a-type recombinant haploid yeast cells comprising a first library of proteins of interest or mutational variants thereof. In FIG. 3 for the purposes of illustration one POI species is represented by single-headed arrows. In this assay many individual cells may each comprise the same species of POI linked to the same molecular barcode. Yeast cell population 302 is a population of α-type recombinant haploid yeast cells comprising a second library of proteins of interest or mutational variants thereof. Yeast cell population 300 and population 302 are combined in liquid culture according to the methods discussed above, interactions between SAPs promote mating between haploid cells to produce diploid yeast cell population 304, and recombination between SAP expression cassettes yields barcode-barcode combinations that are depicted in FIG. 3 as two-headed arrows.

DNA isolation, PCR amplification, and next-generation sequencing yields sequencing reads 306, the abundance of which represents the binding affinity of the POI_{A}-POI_{α} pair. The information available to infer the strength of the interaction is the total number of sequencing reads observed for the POI_{A}-POI_{α} pair.

As an example of the new methods and compositions described herein, FIG. 4 is a schematic of an example of a yeast synthetic agglutination assay for a POI_{A}-POI_{α} pair with multiplexed barcoding, i.e., each POI is linked to a plurality of unique oligonucleotide barcode species. Yeast cell population 400 is a population of a-type recombinant haploid yeast cells comprising a first library of proteins of interest or mutational variants thereof. In FIG. 4, one POI species is represented by single-headed arrows. In this assay, many individual cells may each comprise the same species of POI, but each cell comprising that species of POI should have a unique molecular barcode linked to that POI. Yeast cell population 402 is a population of α-type recombinant haploid yeast cells comprising a second library of proteins of interest or mutational variants thereof. Yeast cell population 400 and population 402 are combined in liquid culture according to the methods discussed above, interactions between SAPs promotes mating between cells haploid cells to produce diploid yeast cell population 404, and recombination between SAP expression cassettes yields barcode-barcode combinations that are depicted in FIG. 4 as two-headed arrows.

As a result of multiplexed barcoding as described herein, each cell of diploid yeast cell population 404 comprises a unique barcode-barcode combination. DNA isolation, PCR amplification, and next-generation sequencing yields sequencing reads 406, where the number of unique barcode-barcode combinations detected represents the number of diploid formation events that occurred during the assay. Binding affinity of the POI_{A}-POI_{α} pair can be accurately inferred from the number of unique barcode-barcode combinations detected. It is important to note that due to variabilities of the assay conditions (i.e. yeast growth rates, PCR amplification, NGS library prep) each unique barcode-barcode combination may be detected by varying numbers of sequencing reads, as shown in FIG. 4.

However, the informative data in the present methods are the number of species of unique barcode-barcode combinations detected rather than the abundance of sequencing reads detected for each barcode-barcode combination. The information available to infer the strength of the POI_{A}-POI_{α} interaction is the total number of unique barcode-barcode combinations detected, representing the number of diploid formation events. Rather than attempt to quantify diploid formation events based on the total number of sequencing reads, as in FIG. 3 and sequencing reads 306, the number of unique barcode-barcode combinations with any sequencing evidence are quantified, as in FIG. 4 and sequencing reads 406. That quantity is used to directly infer the number of diploid yeast formed during the agglutination assay, without regard for the variance introduced during the assay.

Quantifying the number of original diploid formation events, based on quantification of unique barcode-barcode sequences as a proxy for diploid formation, is used as the basis for improved estimation of PPI affinity from sequencing data and more accurate quantification of uncertainty.

### Constructs and Barcodes

As used herein, the term “nucleic acid construct” refers to a contiguous polynucleotide or DNA molecule capable of being integrated into a yeast strain. In some implementations, the nucleic acid construct comprises: (a) a homology arm at the 5′ end of the nucleic acid construct, (b) a first expression cassette comprising a gene encoding a synthetic adhesion protein (SAP) that binds to a cell wall glycosylphosphatidylinositol (GPI) anchored protein, (c) a second expression cassette comprising a first marker, (d) a unique primer binding site, (e) an oligonucleotide molecular barcode, (f) a recombination site, and (g) a homology arm at the 3′ end of the nucleic acid construct. In some implementations, components (a) through (g) of the nucleic acid construct are arranged in a 5′ to 3′ direction on the nucleic acid construct; wherein component (a) is 5′ to component (b) and component (b) is 5′ to component (c) and component (c) is 5′ to component (d) and component (d) is 5′ to component (e) and component (e) is 5′ to component (f) and component (f) is 5′ to component (g) and component (g) is at the 3′ end of the nucleic acid construct.

In some implementations, a nucleic acid construct comprising a first expression cassette encoding a synthetic adhesion protein (SAP) may be integrated into the genome of a yeast cell at a user-defined genomic target site. In other implementations, a nucleic acid construct comprising a first expression cassette encoding a SAP may be, for example, a 2 micron or centromeric plasmid that is not integrated into the yeast genome.

As used herein, the term “expression cassette” refers to a DNA sequence comprising a promoter, an open reading frame, and a terminator. In certain embodiments, the nucleic acid construct comprises one or more expression cassettes. For example, the nucleic acid construct can comprise one, two, three, or more expression cassettes. In certain embodiments, the nucleic acid construct comprises a first expression cassette comprising a fusion gene encoding a first SAP bound to a first cell wall GPI anchored protein, and a second expression cassette comprising a first marker. In some embodiments, the SAP of the first expression cassette of the first nucleic acid construct is fused to the sexual agglutination protein Aga2, and the SAP of the first expression cassette of the second nucleic acid construct is fused to the sexual agglutination protein Aga2, as depicted in FIG. 1 and FIGS. 2A-2C.

In some implementations, the nucleic acid constructs comprise a recombination site. The recombination site allows certain site-specific recombination events once the nucleic acid construct has been integrated into the genomic target region and mating has occurred. In other implementations, the nucleic acid constructs are not integrated into the yeast genome and site-specific recombination events occur between extrachromosomal nucleic acid constructs, e.g., a 2 micron or centromeric plasmid.

In some implementations, the recombination sites are located close to the barcoded SAP expression cassettes and are constructed so that recombination results in a chromosomal translocation that places the two barcodes from each of the first and second nucleic acid constructs that were previously integrated on the same chromosomes of the respective first and second yeast strains onto the same chromosome of the diploid yeast cell. In some implementations, the recombination sites of the first and second nucleic acid constructs are designed so that recombination does not destroy the chromosomes or result in killing the cells. The site-specific recombination events at the recombination sites are controlled by a site-specific recombinase, which catalyzes and mediates the site-specific recombination event between two DNA recombination sites.

In some implementations, one or both of the yeast strains comprises an exogenous recombinase. The recombinase is expressed only in diploid cells following mating. For example, the second recombinant yeast strain can express a transcription factor and the first recombinant yeast strain comprises the exogenous recombinase or the first recombinant yeast strain can express a transcription factor and the second recombinant yeast strain comprises the exogenous recombinase. It is also possible to have both strains comprise the exogenous recombinase and the transcription factor. When expressed in a diploid cell the recombinase mediates recombination between site-specific Cre recombination sites.

In some embodiments, just one of the strains comprises an inducible promoter controlling expression of the exogenous recombinase. To express the recombinase only in the mated diploid cells, an inducible transcription factor, (for example, Zev4), is controllably induced (i.e., Zev4 is activated with beta-estradiol, which permits entry of Zev4 into the nucleus where it then activates the promoter pZ4), and activates transcription from its promoter, which is placed upstream of the recombinase. Thus, adding an inducer (i.e., beta-estradiol) to the mated cells activates expression of the exogenous recombinase and causes a chromosomal translocation in diploids that pairs two barcodes together.

In some implementations, the nucleic acid constructs each comprise a unique primer binding site. The unique primer binding sites are designed to allow amplification with that set of primers that will only amplify a target nucleic acid fragment containing 2 unique barcodes from correctly recombined diploid cells. The target nucleic acid fragment pool is then sequenced, for example, using next generation sequencing.

As used herein, a primer or primer pair refers to an oligonucleotide pair (i.e., a forward and reverse primer), either natural or synthetic, which is capable, upon forming a duplex with a polynucleotide template, of acting as a point of initiation of nucleic acid synthesis and being extended from its 3′ end along the template so that a target nucleic acid fragment is formed. In another implementation, the unique primer binding site of the first nucleic acid construct and the unique primer binding site of the second nucleic acid construct are integrated into the same chromosome and after mating and chromosomal translocation the primer binding sites can be used to amplify a target nucleotide sequence comprising both the unique barcode of the first nucleic acid construct and the unique barcode of the second nucleic acid construct, or a portion of the unique barcode of the first nucleic acid construct and a portion of the unique barcode of the second nucleic acid construct.

In an embodiment, the unique barcode of the first nucleic acid construct and the unique barcode of the second nucleic acid construct are integrated into the same chromosomal locus and after mating and chromosomal translocation are within about 5,000, 4,000, 3,000, 2,000, 1,000, 900, 800, 700, 600, 500, 400, 300, 200, or 100 base pairs. In some embodiments, using next generation sequencing, a paired-end read is used to read the barcodes at either end of a target nucleic acid fragment.

In another implementation, recombination occurs in diploid cells after mating between a first extrachromosomal nucleic acid construct encoding a first SAP coupled to a first oligonucleotide molecular barcode and a second extrachromosomal nucleic acid construct encoding a second SAP coupled to a second oligonucleotide molecular barcode. As a result the unique primer binding site of the first nucleic acid construct and the unique primer binding site of the second nucleic acid construct are on the same molecule and the primer binding sites can be used to amplify a target nucleotide sequence comprising both the unique barcode of the first nucleic acid construct and the unique barcode of the second nucleic acid construct, or a portion of the unique barcode of the first nucleic acid construct and a portion of the unique barcode of the second nucleic acid construct.

In the new methods, the nucleic acid constructs each comprise an oligonucleotide molecular barcode. In some implementations, each barcode is specific to a certain SAP comprising a certain POI. In other implementations, a plurality of unique barcodes sequences is associated with a certain SAP comprising a certain POI. Within a plurality of nucleic acid constructs encoding a single POI, each construct may comprise a unique oligonucleotide molecular barcode, such that a single POI is associated with a diverse plurality of unique oligonucleotide molecular barcodes.

The oligonucleotide molecular barcodes used in the compositions and methods disclosed herein can be, for example, from about 5 nucleotides to 40 nucleotides in length; from about 10 nucleotides to 35 nucleotides in length; from about 15 nucleotides to 30 nucleotides in length; from about 20 nucleotides to 25 nucleotides in length. In some implementations, the oligonucleotide molecular barcodes are 10, 15, 20, 25, or 30 nucleotides in length.

In some embodiments, the barcodes are not specifically chosen. Instead, they are added with degenerate primers that contain a region with random base pairs (for example in a library-by-library screen of SAPs). In some implementations, the oligonucleotide molecular barcodes are synthesized as a degenerate library by nucleic acid synthesis methods well known in the art and combined with a library of constructs encoding a library of POIs by a nucleic acid assembly method, for example, isothermal in vitro recombination.

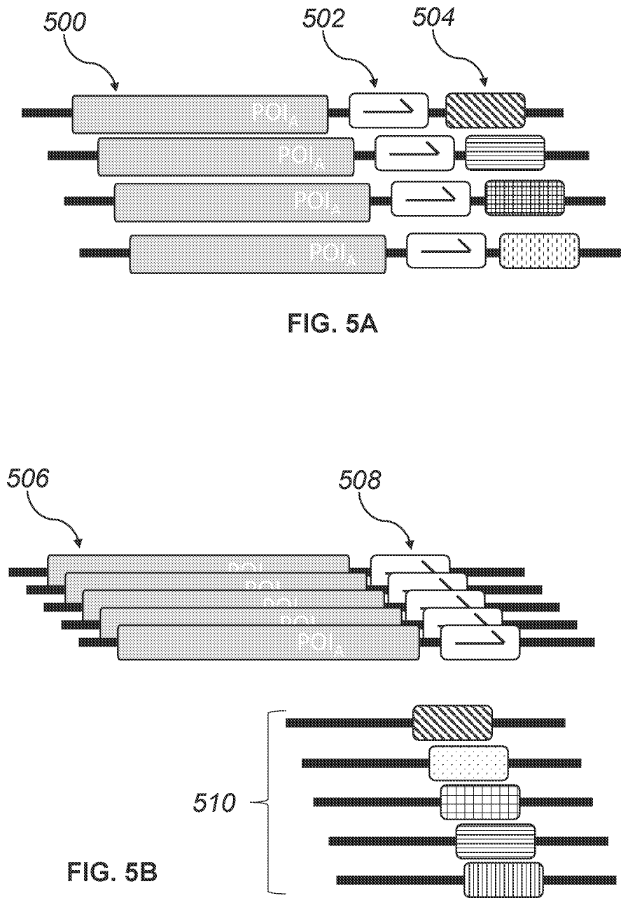

FIG. 5A is a schematic of portions of nucleic acid constructs in which an ORF encoding a POI was synthesized with a plurality of oligonucleotide molecular barcode sequences, with each ORF being linked to a different unique oligonucleotide molecular barcode sequence. FIG. 5A depicts a plurality of nucleic acid constructs comprising an ORF 500 encoding a single POI_{A}, with each construct comprising a unique oligonucleotide molecular barcode sequence 504. Sequence diversity of the oligonucleotide molecular barcode sequences 504 among the different nucleic acid constructs is represented by various patterns in the schematic of FIG. 5A. Primer binding site 502 is used to amplify a unique combined barcode-barcode sequence after cell fusion events as described above. In some implementations, the ORF 500, primer binding site 502, and oligonucleotide molecular barcode sequence 504 can be synthesized by one of several DNA synthesis methods known in the art.

FIG. 5B is a schematic diagram of portions of nucleic acid constructs where a library of oligonucleotide molecular barcode sequences was synthesized separately and assembled with the ORF encoding a POI by isothermal in vitro assembly, yielding a plurality of nucleic acid constructs, each comprising an ORF encoding a POI with each ORF being linked to a different unique oligonucleotide molecular barcode sequence. FIG. 5B depicts a plurality of nucleic acid constructs comprising an ORF 506 encoding a single POI_{A} and a primer binding site 508 that is used to amplify a unique combined barcode-barcode sequence after cell fusion events as described above. A library of oligonucleotide molecular barcode sequences 510 may be synthesized separately by one of several DNA synthesis methods known in the art. Sequence diversity of the oligonucleotide molecular barcode sequences 510 is represented by various patterns in the schematic of FIG. 5B. The resulting library of diverse oligonucleotide molecular barcode sequences can be combined with ORF 506 and primer binding site 508 by isothermal in vitro assembly such that the single POIA encoded by ORF 506 is linked to a diverse plurality of oligonucleotide molecular barcode sequences 510.

In the new methods, the number of observed unique barcode-barcode combinations detected by downstream sequencing for a given POI_{A}-POI_{α} pair relative to the total number of possible barcode-barcode combinations for that POI_{A}-POI_{α} pair is used to estimate the number of diploid formation events that were mediated by the SAPs comprising the POI_{A} and the POI_{α}.

### Proteins of Interest

In some implementations, the compositions and methods comprise a first protein of interest (POI) and a library of second POIs. The library of second POIs may comprise a plurality of user-designated or randomly added mutants of a POI and the wild-type protein. The library of second POIs may comprise a plurality of protein species encoded by a plurality of genes, e.g., human genes. In other implementations, the methods comprise a library of first POIs and a library of second POIs. The plurality of user-designated or randomly added mutants of the first POI or second POI may comprise variants of the POI with 1, 2, 3, 4, 5, 6, 7, 8, 9, 10, or more amino acid substitutions. The amino acid substitutions may be chosen to introduce changes in charge to the POI and/or changes in conformational structure to the POI, and wild-type amino acids may be substituted with natural or non-natural amino acids.

In some implementations, the amino acid substitutions may be generated by site saturation mutagenesis (SSM) to produce an SSM library of POI variants. In some implementations, the library of first POIs or second POIs may be generated by alanine scanning. In some implementations, the library of first POIs or second POIs may be generated by random mutagenesis, such as with error prone PCR, or another method to introduce variation into the amino acid sequence of the expressed protein. The first POI and the library of second POIs, or the library of first POIs and the library of second POIs are assayed for binding affinity according to the methods disclosed herein, such that affinity is measured for interaction between the first POI and each of the plurality of second POIs individually, or between each of the plurality of first POIs and each of the plurality of second POIs individually, in a pair-wise parallelized high-throughput manner.

The library of first POIs or the library of second POIs can include a plurality of user-designated or randomly added mutants of the POI and the wild-type POI. The plurality of user-designated or randomly added mutants of the POI can include variants of the targeting protein with 1, 2, 3, 4, 5, 6, 7, 8, 9, 10, or more amino acid substitutions. The amino acid substitutions may be chosen to introduce changes in charge to the POI or changes in conformational structure to the POI, and wild-type amino acids may be substituted with natural or non-natural amino acids.

In some implementations wherein a library of first POIs is assayed against a library of second POIs for binding affinity, the assay may be a yeast two-hybrid system, synthetic yeast agglutination in liquid culture, or another parallelized high-throughput library-by-library screening method. Binding affinities for the interaction between mutant POIs relative to the binding affinity between wild-type POIs can be measured by any number of methods for quantifying protein binding affinity, including yeast two-hybrid screening, biolayer interferometry, ELISA, quantitative ELISA, surface plasmon resonance, FACS-based enrichment methods, synthetic yeast agglutination in liquid culture, or any other measurement of protein interaction strength. For example, synthetic yeast agglutination in liquid culture is described in U.S. Patent Application Publication No. US 2017/0205421.

In some implementations, the first POI and second POI are full-length proteins. In other implementations, the first POI and second POI are truncated proteins. In other implementations, the first POI and second POI are fusion proteins. In other implementations, the first POI and second POI are tagged proteins. Tagged proteins include proteins that are epitope tagged, e.g., FLAG-tagged, HA-tagged, His-tagged, Myc-tagged, among others known in the art. In some implementations, the first POI is a full-length protein and the second POI is a truncated protein. The first POI and second POI may each be any of the following: a full-length protein, truncated protein, fusion protein, tagged protein, or combinations thereof.

In some implementations, the first POI is an antibody or truncated portion of an antibody polypeptide. In other implementations, the library of first POIs is a library of antibodies, truncated antibody polypeptides, or a library of antibody mutants generated by site saturation mutagenesis, alanine scanning, or other methods of introducing a plurality of amino acid variants well known in the art. Antibodies, also known as immunoglobulins, are relatively large multi-unit protein structures that specifically recognize and bind a unique molecule or molecules. For most antibodies, two heavy chain polypeptides of approximately 50 kDA and two light chain polypeptides of approximately 25 kDA are linked by disulfide bonds to form a larger Y-shaped multi-unit structure. Variable and hypervariable regions representing amino-acid sequence variability at the tips of the Y-shaped structure confer specificity for a given antibody to recognize its target.

In some implementations, the first POI is a single-chain variable fragment (scFv), a fusion protein of the variable regions of the heavy (VH) and light chains (VL) of an immunoglobulin connected by short linker peptides. In some implementations, the library of first POIs is a library of scFvs or a library of scFvs mutants generated by site saturation mutagenesis, alanine scanning, or other methods of introducing a plurality of amino acid variants well known in the art.

In some implementations, the first POI is an antigen-binding fragment (Fab), a region of an antibody that binds to an antigen. A Fab may comprise one constant and one variable domain of each of the heavy and the light chain, and includes the paratope region of the antibody. In some implementations, the library of first POIs is a library of Fabs or a library of Fab mutants generated by site saturation mutagenesis, alanine scanning, or other methods of introducing a plurality of amino acid variants well known in the art.

In some implementations, the first POI may be a portion of a single domain antibody, or VHH, the antigen-binding fragment of a heavy chain only antibody. A VHH comprises one variable domain of a heavy-chain antibody. In some implementations, the library of first POIs is a library of VHHs or a library of VHH mutants generated by site saturation mutagenesis, alanine scanning, or other methods of introducing a plurality of amino acid variants well known in the art.

In some implementations, the first POI is an E3 ubiquitin ligase. In other implementations, the library of first POIs is a library of E3 ubiquitin ligases or a library of E3 ubiquitin ligase mutants generated by site saturation mutagenesis, among other methods. E3 ubiquitin ligases include MDM2, CRL4^{CRBN}, SCF_{β-TrCP}, UBE3A, and other species that are well known in the art. E3 ubiquitin ligases recruit the E2 ubiquitin conjugating enzyme that has been loaded with ubiquitin, recognize its target protein substrate, and catalyze the transfer of ubiquitin molecules from the E2 to the protein substrate for subsequent degradation by the proteasome complex.

In some implementations, the second POI is a target protein comprising a degron. In other implementations, the library of second POIs is a library of polypeptides comprising degrons or a library of polypeptides comprising degron mutants generated by site saturation mutagenesis, among other methods. A degron is a portion of a polypeptide that mediates regulated protein degradation, in some cases by the ubiquitin proteasome system. Degrons may include short amino acid motifs, post-translational modifications, e.g., phosphorylation, structural motifs, and/or sugar modifications.

In some implementations wherein the second POI is a degron, the degron may be fluorescently tagged, i.e., by expressing the degron as a fusion protein that includes a genetically encoded fluorescent tag, e.g., green fluorescent protein (GFP), red fluorescent protein (RFP), mCherry, M Scarlet, tdTomato, among others.

In some implementations, the first POI is E3 ubiquitin ligase. The library of second POIs may comprise, for example, polypeptide substrate species known in the art to be associated with the E3 ubiquitin ligase. The second library of POIs may further comprise, for example, previously known full-length mapped E3 ubiquitin ligase substrate domains; high-throughput oligonucleotide-encodable truncated E3 ubiquitin ligase substrates; E3 ubiquitin ligase substrate species that have been modified by site saturation mutagenesis; previously defined degron motifs; or computationally-predicted degron motifs. The library of second POIs may comprise a plurality of user-designated mutants of a polypeptide substrate and the wild-type polypeptide substrate. The plurality of user-designated mutants of a POI may comprise variants of the POI with 1, 2, 3, 4, 5, 6, 7, 8, 9, 10, or more amino acid substitutions. The amino acid substitutions may be generated by site saturation mutagenesis. The first POI and the library of second POIs may be assayed for binding affinity, such that affinity is measured for interaction between the first POI and each of the plurality of user-designated mutants of the second POI individually, in a pair-wise parallelized high-throughput manner.

### Yeast Strains and Culture Conditions

For library-by-library screening of PPIs according to the methods disclosed herein, yeast sexual agglutination is re-engineered. For example, the natural protein-protein interaction between native sexual agglutination proteins in S. cerevisiae, binding of which is essential for mating in liquid culture, is replaced by the interaction between two proteins of interest expressed as multiplex barcoded synthetic adhesion proteins on the surface of recombinant haploid yeast cells. To construct recombinant yeast strains for use in the methods disclosed herein, isogenic fragments for yeast transformation or plasmid assembly can be PCR amplified from existing plasmids, yeast genomic DNA, animal or human cDNA, animal or human genomic DNA, cDNA gel extracted from a plasmid digest, or commercially synthesized by conventional DNA synthesis methods. Plasmids can be constructed by isothermal assembly and verified with Sanger sequencing, which may also be used to identify the diverse plurality of oligonucleotide molecular barcodes sequences that are linked to each ORF encoding a SAP or SAP variant.

In some implementations, a MATa haploid strain optimized for surface display, e.g. EBY100, can be used as a parent strain. A parent MATalpha haploid surface display strain can be constructed with mating, sporulation, tetrad dissection, and screening with selectable markers. Isogenic chromosomal integrations can be performed by digesting a plasmid with Pmel followed by a standard lithium acetate yeast transformation protocol. SSM libraries of SAPs may be transformed into yeast using nuclease assisted chromosomal integration. Parent strains containing a landing pad, e.g., a Scel landing pad, can be grown for 6 hours in galactose media prior to transformation. Recycling of the URA3 gene may be accomplished by growing a strain to saturation without URA selection and plating on 5-FOA.

In some implementations, an isogenic strain may be constructed individually and the plurality of oligonucleotide molecular barcodes associated with each SAP may be determined with Sanger sequencing or next generation sequencing. A library of yeast strains, all of the same mating type, displaying unique barcoded SAP wherein each ORF encoding an SAP is linked to a plurality of oligonucleotide molecular barcode sequences, may be produced. Each haploid strain in the library may be individually grown to saturation, evaluated for surface expression strength as described previously, and mixed in equal volumes. After growing to saturation, cells may be harvested by centrifugation and lysed by heating to 70° C. for 5 minutes in 200 mM LiOAc and 1% SDS. Cellular debris may be removed and incubated at 37° C. for 4 hours with 0.05 mg/mL RNase A. An ethanol precipitation may be performed to purify and concentrate the genomic DNA. A primary qPCR may be performed to amplify the barcode region with standard adaptors and the PCR product is used as a template for a secondary qPCR to attach an index barcode and standard Illumina adaptors for next-generation sequencing. This fragment may be gel extracted, quantified with a Qubit, and analyzed on a commercially available next generation sequencing platform.

In some implementations, for large-scale library matings constructed with nuclease assisted chromosomal integration, mating type libraries may be grown separately to saturation in 3 mL YPD media. 1 mL of the MATa culture and 2 mL of the MATalpha culture may be mixed and genome prepped according to standard conditions. This genomic DNA may be used as a template for two separate qPCR reactions, one to amplify the MATa expression cassette and barcode and the other to amplify the MATalpha expression cassette and barcode. A secondary PCR may be used to add different sequencing index barcodes and Illumina adaptors. These fragments may be sequenced using a commercial next-generation sequencing platform, e.g., Illumina MiSeq. 2.5 μL of the MATa culture and 5 μL of the MATalpha culture may be combined in 3 mL of YPD media and treated the same as for the small-scale batched mating.

In some implementations, for small scale libraries, the plurality of oligonucleotide molecular barcode sequences for each SAP are determined with Sanger sequencing after the synthesized library of barcodes has been assembled with the nucleic acid construct comprising the SAP expression cassettes (see, e.g., FIG. 5B). For large-scale libraries, a next generation sequencing run may be required to map each SAP to the associated plurality of oligonucleotide molecular barcode sequences and to determine the starting concentration of each SAP expressing strain. Next generation sequencing of fragments amplified from diploid genomic DNA after SAP-mediated fusion events provides the identity of combined unique barcode-barcode pairs occurring in the same fragment (see, e.g., FIG. 4), with each unique combined sequence representing an individual mating event.

In some implementations, a multiplexed SAP barcoding and recombination scheme may be used to analyze whole protein interaction networks in a single assay. Single MATa and MATα parent strains, for example yNGYSDa and yNGYSDa, may be constructed and multiplex-barcoded SAP cassettes, or plasmids carrying multiplex-barcoded SAP cassettes, may be transformed into the strains according to a conventional yeast transformation protocol. In addition to the knockout of Sag1 in both parent strains and complementary lysine and leucine markers, yNGYSDa contains a CRE recombinase expression cassette with an inducible promoter, pZ4, and constitutively expresses ZEV4, an activator of the pZ4 promoter with an estradiol binding domain for nuclear localization.

SAP cassettes can be assembled in a standardized vector, for example, pNGYSDa or pNGYSDa, for integration into a corresponding yeast parent strain. In addition to the surface expression cassette, each vector backbone may contain one or more of the following: a mating type specific florescent reporter cassette, one of a plurality of oligonucleotide molecular barcode sequences, a mating type specific primer binding site, and a lox recombination site. Upon cell-cell fusion and mating, β-Estradiol (βE) can be added to induce CRE recombinase expression in fused diploid cells, consolidating the barcodes from each haploid chromosome so that next generation sequencing can be used to identify unique barcode-barcode combinations, each representing a unique individual cell-cell fusion event mediated by interacting SAP pairs (see, FIGS. 1-4).

The number of unique cell-cell fusion events for each SAP interaction in the network is estimated from the number of unique combined barcode sequences detected by sequencing, according to methods described in further detail below, providing a relative interaction strength for each PPI in the network. Using the estimated number of diploid yeast formed for each pair of POI_{A}-POI_{α} pair, and adjusting for the proportion of the input libraries made up by each POI, PPI affinity can be estimated by reference to a set of PPI standards with known affinities, i.e., positive and negative controls.

Many of the yeast strains described for use in the methods disclosed herein may undergo multiple transformations. Displayer strains compatible with the CRE recombinase assay, for example, may require the integration of Agal under the control of a constitutive promoter, the knockout of a native sexual agglutinin protein, the integration of a fluorescent reporter, the integration of CRE recombinase and GAVN or of HygMX and ZEV4, and the integration of a plurality of barcoded surface expression cassettes with a lox site. For each integration, a plasmid may be constructed that contains the required yeast cassette, an E. coli resistance marker and origin of replication, and 5′ and 3′ regions of homology to the yeast genome for integration.

### Statistical Analysis

After next-generation sequencing and quantification of the number of unique barcode-barcode combinations detected among the sequencing reads, the number of diploid formation events can be inferred. The inference is essentially equivalent to the classic “balls into bins” problem in probability theory: if one throws an unknown quantity of balls in n bins, then quantifies how many bins contain balls, how many balls were thrown? In the scenario where the number of bins vastly exceeds the number of balls and the balls are thrown at random, the estimated number of balls is simply equivalent to the number of bins that have balls in them.

Applied here, the “bins” are equivalent to the number of possible unique barcode-barcode combinations, i.e., the number of POI_{A} barcodes multiplied by the number of POI_{α} barcodes. The number of “balls in bins” is equivalent to the number of unique barcode-barcode combinations detected after sequencing. When the number of possible unique barcode-barcode combinations vastly exceeds the number of diploid formation events that are expected to occur, which is expected to be the case for library-by-library PPI assays described here, the number of diploid formation events can be estimated as equivalent to the number of unique barcode-barcode combinations detected after sequencing. To account for the possibility that some barcode pairs may have been paired more than once, which is unlikely here, one can estimate the total number of diploid formation events by solving for the Poisson rate parameter that matches the observed proportion of barcode pairs:

estimated diploids formed = # of possible barcode pairs * −In(proportion
of unobserved barcode pairs)

Using the estimated number of diploid yeast formed for each pair of POI_{A}-POI_{α} pair, and adjusting for the proportion of the input libraries made up by each POI, PPI affinity can be estimated by reference to a set of PPI standards with known affinities, i.e., positive and negative controls.

### EXAMPLES

The compositions and methods disclosed herein are further described in the following examples, which do not limit the scope of the compositions and methods described in the claims. A number of embodiments of the invention have been described. Nevertheless, it will be understood that various modifications may be made without departing from the spirit and scope of the invention.

### Example 1—Synthetic Yeast Agglutination with Multiplexed Barcoding

This example demonstrates a synthetic yeast agglutination assay in liquid culture for library-on-library characterization of protein-protein interactions (PPIs) that combines yeast surface display and sexual agglutination to link protein binding to the mating of S. cerevisiae, utilizing the multiplexed barcoding approach described herein, where each SAP of the libraries of SAPs was linked to a plurality of unique oligonucleotide molecular barcodes. After a given pair of SAPs that interact mediated mating between an a-type recombinant haploid yeast cell and an α-type recombinant haploid yeast cell, CRE recombinase expression was induced in diploids and a βEa recombination event at lox sites consolidated both SAP-Aga2 fusion expression cassettes onto the same chromosome resulting in the barcode linked to the first SAP and the barcode lined to the second SAP in proximity to each other.

In this example, each SAP of a SAP library was linked to many unique barcodes, so that each diploid fusion event and subsequent recombination event produced a unique barcode-barcode combination. A single fragment containing both barcodes was then amplified by PCR with primers annealing to P_{f} and Pᵣ (primers specific to the primers from the first and second nucleic acid constructs integrated at the genomic target site) and sequenced to identify the interacting SAP pair.

The multiplexed barcoding and recombination scheme was developed for the analysis of whole protein interaction networks in a single liquid culture. A library of 36,000 POIs—mutational variants of an antibody—was assayed against a library of 500 POIs—antibody targets of interest for assessing cross-reactivity. Single MATa and MATα parent strains, yNGYSDa and yNGYSDα, were constructed. Multiplexed barcoded SAP expression cassettes were assembled by combining an SAP library with a library of unique oligonucleotide molecular barcodes by isothermal assembly.

Multiplex barcoded SAP cassettes were transformed into the yeast strains, with a seven unique oligonucleotide molecular barcodes linked to each SAP. In addition to the knockout of Sag1 in both parent strains and complementary lysine and leucine markers, yNGYSDa contained a CRE recombinase expression cassette with an inducible promoter, pZ4. yNGYSDa constitutively expressed ZEV4, an activator of the pZ4 promoter with an estradiol binding domain for nuclear localization. SAP cassettes were assembled in one of two standardized vectors, pNGYSDa or pNGYSDa, for integration into the corresponding yeast parent strain. In addition to the surface expression cassette, each vector backbone contained a mating type specific florescent reporter cassette, a unique randomized ten-nucleotide barcode with seven unique barcodes linked to each SAP, a mating type specific primer binding site, and a lox recombination site.

Upon mating, the addition of β-Estradiol (βE) induced CRE recombinase expression in diploid cells, consolidating the barcodes from each haploid chromosome so that next-generation sequencing could be used to identify the number of unique diploid formation events that occurred during the assay. The number of unique barcode-barcode combinations for each SAP pair was quantified from the unique NGS sequencing reads, providing a highly accurate estimate of the number of original diploid formation events via digital quantitation.

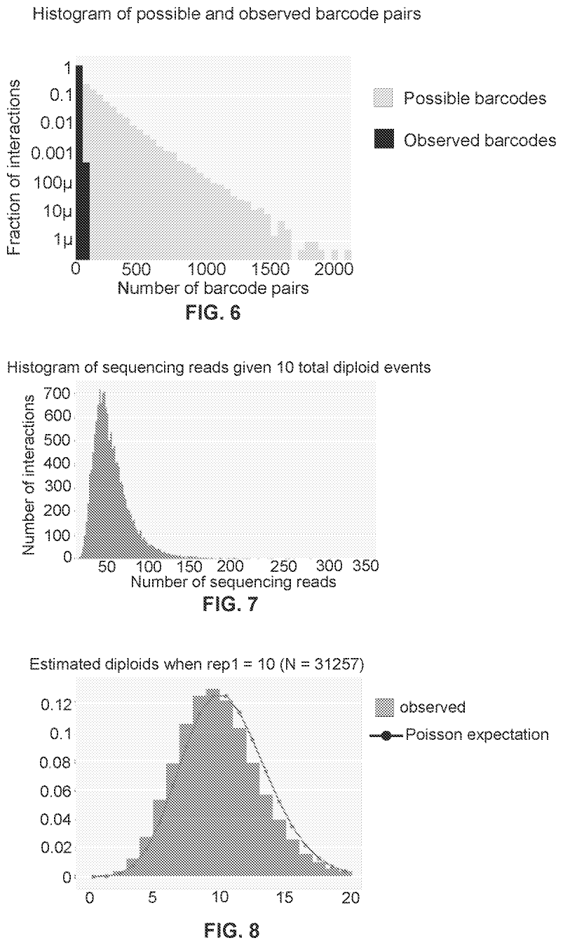

FIG. 6 is a plot of data from this example and shows a histogram of possible and observed barcode pairs for each pair of POIs with any observed sequencing data. In this example, for POI_{A}-POI_{α}, there were approximately 48 potential unique barcode-barcode combinations, with 7 unique barcodes linked to each POI. The overwhelming majority of POI pairs have very few barcode pairs observed, with a mean of 0.5 barcodes observed across POI_{A}-POI_{α} pairs. However, the distribution of potential barcodes pairs is shifted substantially toward higher values. This example illustrates a situation where the multiplexed barcoding scheme is particularly useful in improving quantitative accuracy.

FIG. 7 shows the distribution of sequencing reads observed among POI pairs where 10 diploid yeast were estimated with high confidence to have been formed during the synthetic yeast agglutination assay of this example. These are POI pairs for which exactly 10 unique barcode-barcode combinations were observed in the sequencing data where at least 200 possible barcode-barcode combinations for each POI pair were expected. This figure demonstrates that as expected, the processes of yeast growth, PCR amplification, and next-generation sequencing introduces substantial variation in the final number of sequencing reads generated for each POI pair, as the plot shows that there is a wide distribution of the number of sequencing reads among unique POI pairs.

From the digital quantitation enabled by the multiplexed barcoding of POIs, one can infer with high confidence that 10 diploids formed for each POI pair. Without the multiplexed barcoding, this inference would have been skewed to indicate that POI pairs with higher numbers of sequencing reads had higher affinity, which is not necessarily the case.

FIG. 8 confirms that the multiplexed barcoding method can accurately estimate the number of diploid yeast formed during mating for each POI pair that binds. If the number of diploid yeast formed during the assay is being correctly estimated, then the variance observed when repeating the experiment should follow a roughly Poisson distribution, μ=the number of estimated diploids formed during the assay for a POI pair. FIG. 8 shows the distribution of estimated diploids for POI pairs that had 10 estimated diploids formed during the experiment of this example. The assay was performed two additional times as biological replicates. As shown in FIG. 8, the close agreement between empirical observation and statistical expectation indicates that the original estimation of 10 diploid events in the first replicate was highly accurate.

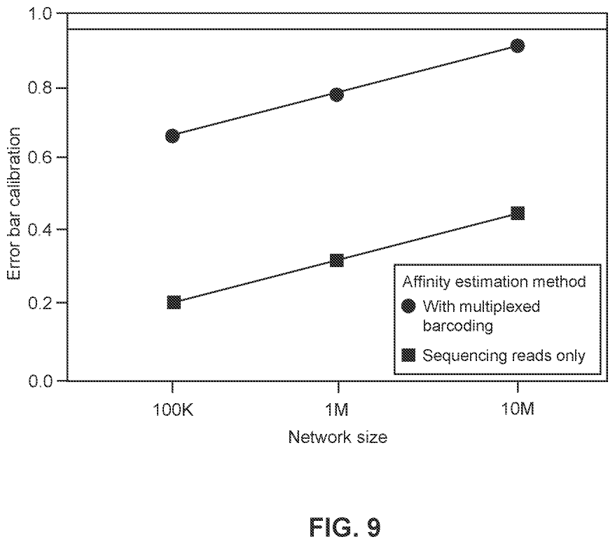

To demonstrate the improvement of multiplex barcoding of SAPs over simplex barcoding of SAPs for the estimation of PPI affinities further, we calculated confidence intervals for PPI networks of increasing size. FIG. 9 shows the impact of multiplexed barcoding on the estimation of uncertainty in PPI affinity. Four networks of POI_{A}-POI_{α} interactions, of various overall network size, were measured using the synthetic yeast agglutination assay. All networks shared a core set of 14 standard positive and negative controls, 42 additional variants of positive controls, 33 anti-SARS-COV2 antibodies, 60 RBD domains from multiple SARS-COV2 strains and variants, 47 anti-flaA antibodies, and five flaA variants. Larger networks were expanded from this core set of interactions by adding additional variants of the antibodies and related targets.

For each network, correct behavior for uncertainty quantification would comprise 95% of measurements from the smallest network (high confidence estimates, represented by horizontal bar at the top of the plot depicted in FIG. 9) falling within the nominal 95% confidence interval calculated for the larger network.

Confidence intervals calculated using multiplexed barcoding captured 70-90% of the data. These results indicate that the confidence intervals are slightly too narrow, but are close to the nominal target of 95%. In contrast, the method utilized previously without multiplexed barcoding (using +/−2 empirical standard deviations among biological replicates to construct confidence intervals) results in confidence intervals that are much too narrow, with only 20-40% of high confidence measurements falling within a nominal 95% confidence window. This example illustrates how assigning multiplexed barcodes to each POI can substantially improve the ability to quantify the uncertainty in PPI affinity measurements derived from the synthetic yeast agglutination assay.

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

1. A method for quantifying unique cell fusion events, the method comprising:
providing a first quantity of cells, wherein each cell of the first quantity of cells comprises an exogenous nucleic acid vector of a first library of exogenous nucleic acid vectors, wherein each of the exogenous nucleic acid vectors in the first library comprises a first open reading frame (ORF) linked to an oligonucleotide molecular barcode sequence selected from a first plurality of oligonucleotide molecular barcode sequences;
providing a second quantity of cells, wherein each cell of the second quantity of cells comprises an exogenous nucleic acid vector of a second library of exogenous nucleic acid vectors, wherein each of the exogenous nucleic acid vectors in the second library comprises a second ORF linked to an oligonucleotide molecular barcode sequence selected from a second plurality of oligonucleotide molecular barcode sequences;
combining the first quantity of cells and the second quantity of cells in a liquid medium to produce a culture;
growing the culture for a time and under conditions sufficient to enable fusion events to occur between cells of the first quantity of cells and cells of the second quantity of cells to produce a plurality of fused cells, wherein a recombination event occurs between the first exogenous nucleic acid vector and the second exogenous nucleic acid vector within the fused cells to produce combined oligonucleotide molecular barcode sequences;
sequencing combined oligonucleotide molecular barcode sequences from the culture;
determining, for each pair of first ORF and second ORF, a first number of unique pairs of first and second oligonucleotide molecular barcode sequences within the combined oligonucleotide molecular barcodes observed in the culture;
determining, for each pair of first ORF and second ORF, a second number of possible combined oligonucleotide molecular barcode sequences; and
calculating an estimated number of unique fusion events in the culture based on the first number and second number.

2. The method of claim 1, wherein the first quantity of cells and the second quantity of cells are yeast cells.

3. The method of claim 1, wherein the first quantity of cells are a-type haploid yeast cells and the second quantity of cells are α-type haploid yeast cells.

4. The method of claim 2, wherein the first ORF encodes a protein of interest “a” (POIa) and the second ORF encodes a protein of interest “α” (POIα).

5. The method of claim 4, wherein each ORF encoding a POIa is operably linked to an oligonucleotide molecular barcode sequence selected from the first plurality of oligonucleotide molecular barcode sequences, and each ORF encoding a POIα is operably linked to an oligonucleotide molecular barcode sequence selected from the second plurality of oligonucleotide molecular barcode sequences.

6. The method of claim 4, wherein each POIa is expressed on a surface of a cell of the first quantity of cells and each POIα is expressed on a surface of a cell of the second quantity of cells.

7. The method of claim 3, wherein at least one of the first quantity of cells or the second quantity of cells has been rendered incapable of mating according to any native sexual agglutination process such that the first quantity of recombinant haploid yeast cells and the second quantity of recombinant haploid yeast cells are not capable of mating according to any native sexual agglutination process.

8. The method of claim 4, wherein each POIa and each POIα are synthetic adhesion proteins (SAPs).

9. The method of claim 4, wherein each POIₐ and each POI_{α} are either
i) a fusion protein bound to a cell wall glycosylphosphatidylinositol (GPI) anchored protein residing on a surface of a portion of the first quantity of recombinant haploid yeast cells or the second quantity of haploid yeast cells; or
ii) a glycosylphosphatidylinositol (GPI) anchored fusion protein residing on the surface of a portion of the first quantity of haploid yeast cells or the second quantity of haploid yeast cells.

10. The method of claim 1, wherein the first plurality of oligonucleotide molecular barcode sequences comprises 3 or more unique oligonucleotide molecular barcode sequences, the second plurality of oligonucleotide molecular barcode sequences comprises 3 or more oligonucleotide molecular barcode sequences, or both the first plurality of oligonucleotide molecular barcode sequences and the second plurality of oligonucleotide molecular barcode sequences each comprises 3 or more unique oligonucleotide molecular barcode sequences.

11. The method of claim 1, wherein the first plurality of oligonucleotide molecular barcode sequences comprises 10 or more unique oligonucleotide molecular barcode sequences, the second plurality of oligonucleotide molecular barcode sequences comprises 10 or more oligonucleotide molecular barcode sequences, or both the first plurality of oligonucleotide molecular barcode sequences and the second plurality of oligonucleotide molecular barcode sequences each comprises 10 or more unique oligonucleotide molecular barcode sequences.

12. The method of claim 1, wherein the first plurality of oligonucleotide molecular barcode sequences comprises 100 or more unique oligonucleotide molecular barcode sequences, the second plurality of oligonucleotide molecular barcode sequences comprises 100 or more oligonucleotide molecular barcode sequences, or both the first plurality of oligonucleotide molecular barcode sequences and the second plurality of oligonucleotide molecular barcode sequences each comprises 100 or more unique oligonucleotide molecular barcode sequences.

13. The method of claim 1, wherein the first plurality of oligonucleotide molecular barcode sequences comprises 1000 or more unique oligonucleotide molecular barcode sequences, the second plurality of oligonucleotide molecular barcode sequences comprises 1000 or more oligonucleotide molecular barcode sequences, or both the first plurality of oligonucleotide molecular barcode sequences and the second plurality of oligonucleotide molecular barcode sequences each comprises 1000 or more unique oligonucleotide molecular barcode sequences.

14. The method of claim 1, wherein the second number of possible oligonucleotide molecular barcode pairs is 9 or greater.

15. The method of claim 1, wherein the second number of possible oligonucleotide molecular barcode pairs is 100 or greater.

16. The method of claim 1, wherein the second number of possible oligonucleotide molecular barcode pairs is 10,000 or greater.

17. The method of claim 4, wherein the library of POIas comprises 10 or more POIas and/or the library of POIαs comprises 10 or more POIαs.

18. The method of claim 4, wherein the library of POIas comprises 100 or more POIas and/or the library of POIαs comprises 100 or more POIαs.

19. The method of claim 4, wherein the library of POIas comprises 1000 or more POIas and/or the library of POIαs comprises 1000 or more POIαs.

20. The method of claim 4, wherein the library of POIas comprises 10,000 or more POIas and/or the library of POIαs comprises 10,000 or more POIαs.

21. The method of claim 1, wherein the first exogenous nucleic acid vector and the second exogenous nucleic acid vector each further comprises a unique primer binding site, a recombination site, and a selectable marker.

22. The method of claim 1, wherein each cell of the first quantity of cells and each cell of the second quantity of cells further comprises an exogenous recombinase.

23. The method of claim 22, wherein the exogenous recombinase mediates the recombination event.

24. The method of claim 1, wherein sequencing a portion of the first oligonucleotide molecular barcode sequence and a portion of the second oligonucleotide molecular barcode sequence yields a plurality of sequencing reads, each sequencing read comprising a portion of the first oligonucleotide molecular barcode sequence and a portion of the second oligonucleotide molecular barcode sequence.

25. The method of claim 7, wherein
i) each cell of the first quantity of cells lacks either a functional Agal or a functional Aga2 protein, or
ii) each cell of the second quantity cells lacks a functional Sagl protein, or
iii) each cell of the first quantity of cells lacks either a functional Agal or a functional Aga2 protein and each cell of the second quantity cells lacks a functional Sagl protein.
